(19) United States
(12) Patent Application Publication
(10) Pub. No.: US 2021/0085237 A1
(43) Pub. Date: Mar. 25, 2021

(54) Textile Sensor Assemblies

(71) Applicant: The Trustees of Columbia University in the City of New York, New York, NY (US)

(72) Inventors: Xiaofan Jiang, New York, NY (US); Ji Jia, New York, NY (US)

(21) Appl. No.: 17/061,873

(22) Filed: Oct. 2, 2020

Related U.S. Application Data
(63) Continuation of application No. PCT/US2019/027920, filed on Apr. 17, 2019.
(60) Provisional application No. 62/658,654, filed on Apr. 17, 2018.

(51) Int. Cl. A61B 5/00

(52) U.S. Cl. CPC A61B 5/4266; A61B 5/1118; A61B 5/6802; A61B 5/742

(57) ABSTRACT

Disclosed is a sensor that includes a first textile assembly having a first conductive textile element and a first outer sheath that surrounds the first conductive textile element. The first outer sheath is formed from a first non-conductive material that is configured to transport moisture through the first outer sheath. The sensor includes a second textile assembly having a second conductive textile element and a second outer sheath that surrounds the second conductive textile element. The second outer sheath is formed from a second non-conductive material that is configured to transport moisture through the second outer sheath. The first and second outer sheaths are in contact with each other so as to maintain separation between the first conductive textile element and the second conductive textile element along a length of the sensor.

### RELATED APPLICATION

The present application claims priority to and the benefit of International patent application no. PCT/US2019/027920 (filed Apr. 17, 2019), which claims priority to and the benefit of U.S. patent application No. 62/658,654 (filed Apr. 17, 2018), the entireties of which are incorporated herein by reference for any and all purposes.

### TECHNICAL FIELD

The present disclosure relates to textile sensor assemblies.

### BACKGROUND

Monitoring of perspiration level monitoring enables individual physical condition estimation, e.g., personal comfort level, health condition, exercise monitoring, and hazardous environment detection. For example, for elderly or patient who cannot effectively express their feelings, monitoring their sweat level can help nurse further understand their physical comfort. Prior work on perspiration (sweat) sensing has explored various approaches to design the sensor. However, they either require the user to be static or require the user to wear the adhesive sensor directly on the skin, which limits users' mobility and reduces comfort.

### SUMMARY

An embodiment of the present disclosure is a sensor. The sensor includes a first textile assembly having a first conductive textile element and a first outer sheath that surrounds the first conductive textile element. The first outer sheath is formed from a first non-conductive material that is configured to transport moisture through the first outer sheath. The sensor includes a second textile assembly having a second conductive textile element and a second outer sheath that surrounds the second conductive textile element. The second outer sheath is formed from a second non-conductive material that is configured to transport moisture through the second outer sheath. The first and second outer sheaths are in contact with each other so as to maintain separation between the first conductive textile element and the second conductive textile element along a length of the sensor.

In another embodiment, the sensor further includes an outer cover that at least partially encases the first and second textile assemblies.

In another embodiment, the outer cover is a knitted structure, a woven structure, or a braided structure.

In another embodiment, the first non-conductive material and the second non-conductive material are braided.

In another embodiment, the first non-conductive material and the second non-conductive material are knitted.

In another embodiment, one of the first non-conductive material and the second non-conductive material is knitted.

In another embodiment, one of the first non-conductive material and the second non-conductive material is braided.

In another embodiment, the first non-conductive material and the second non-conductive material comprise absorbent fibers. The non-conductive material could be any absorbent fibers, such as viscose rayon or any fiber that has the ability to transport moisture relatively quickly, e.g. trilobal fibers, 4DG fibers, and the like.

In another embodiment, the first non-conductive material and the second non-conductive material comprise moisture transporting fibers.

In another embodiment, the first non-conductive material and the second non-conductive material comprise cotton fibers.

The present disclosure thus includes novel wearable sensor system that measures an individual's sweat level. The system consists of multiple covered conductive elements that formed into a sensor assembly. The structure allows robust perspiration level estimation despite the sensor distortion or the motion of the person. When the person sweats, and the moisture level of the cotton cover changes, the resistance between the two conductive threads changes with it. One can characterize the relation between the volume of the sweat and the change of the resistance as a reference to measure sweat levels. Using this characterization in one example, non-limiting embodiment, an average estimation error of 0.4 levels was achieved when compared to a 5-level scale of human comfort.

Also provided are sweat level monitoring systems, comprising: a sensor comprising: a first textile assembly having a first conductive element and a first outer sheath that surrounds the first conductive element, the first outer sheath formed from a first non-conductive material, and a second textile assembly having a second conductive element and a second outer sheath that surrounds the second conductive element, the second outer sheath formed from a second non-conductive material, the first and second sheaths maintaining separation between the first conductive element and the second conductive element, the sensor configured to detect a voltage value between the first conductive element and the second conductive element; and a processor configured to: receive the detected voltage value from the sensor, and map the detected voltage value to a moisture characteristic.

Further provided are methods for determining a moisture characteristic with a moisture characteristic monitoring system, the methods comprising: detecting, via a sensor, a voltage value, wherein the sensor comprises a first textile assembly and a second textile assembly, the first textile assembly having a first conductive element and a first outer sheath that surrounds the first conductive element, the first outer sheath formed from a first non-conductive material, and the second textile assembly having a second conductive element and a second outer sheath that surrounds the second conductive element, the second outer sheath formed from a second non-conductive material, wherein the detected voltage value is detected between the first conductive element and the second conductive element; and determining the moisture characteristic based on the detected voltage value.

Additionally provided are methods, comprising: with a first textile assembly having a first conductive element and a first outer sheath that surrounds the first conductive element, the first outer sheath formed from a first non-conductive material that is configured to transport moisture through the first outer sheath; and a second textile assembly having a second conductive element and a second outer sheath that surrounds the second conductive element, the second outer sheath formed from a second non-conductive material that is configured to transport moisture through the second outer sheath, the first and second outer sheaths maintaining a separation between the first conductive element and the second conductive element along a length of the sensor, detecting a voltage related to an amount of moisture that places the first conductive element into electrical communication with the second conductive element.

### BRIEF DESCRIPTION OF THE DRAWINGS

FIG. 1 shows a textile sensor embedded in a garment and sensing swear on a according to an embodiment of the present disclosure.

FIG. 2 is a block diagram of sensor system according to an embodiment of the present disclosure.

FIG. 3 illustrates a sectional view of a textile sensor according to an embodiment of the present disclosure.

FIG. 4 illustrates a circuit for the textile sensor shown in FIGS. 1 to 3.

FIG. 5 illustrates a test assembly used to characterize a textile sensor according to an embodiment of the present disclosure.

FIG. 6A is a sectional view of a textile sensor, illustrating a simplified model of sweat in contact with the sensor.

FIG. 6B is top sectional view of the textile sensor shown in FIG. 6A.

FIG. 7 is a graph illustrating sensor thread resistance as function of solution quantity.

FIG. 8 is schematic view of the sensing system in operation, illustrating how the sensor measures a moisture characteristic (e.g., a moisture level) in voltage of the textile sensors and sends this data to the server, which then calculates the moisture level in mg and estimates each user's perceived sweat scale based on personal historical data. The user can check the measured sweat level as well as their estimated perceived sweat scale on their mobile app.

FIGS. 9A-9C illustrate characterization of the sensor with motion causing sensor bending.

FIG. 10 is a graph showing measured voltage over time for both single threads and multiple threads illustrating a more stable reading for single threads, which can be caused by human motion and sensor bending.

FIG. 11 is graph showing the effect of different levels of motion applied to the sensor as discussed in FIGS. 9A-9C. One can observe that under static, slow and fast motion, the sensor shows similar profiling on the evaporation process. This indicates that the disclosed sensor is robust to the motion causing the sensor's distortion.

FIGS. 12A-12C illustrates the sensor characterization with sharp static sensor bending. The subfigures 12A, 12B, and 12C shows the bending angle of 30, 60, and 90 respectively.

FIG. 13 is graph showing the effect of bending angle on evaporation, which illustrates that the present textile sensor is robust to the bending distortion.

FIG. 14 illustrates a graph showing effect of solution density on evaporation. One can observe that for different solution densities below 4%, the sensor shows similar profiling on the evaporation process, while when the solution density is as high as 4%, the voltage increase rate (resistance decrease rate) slows down. This could be caused by the combination of a high density of the electrolytes and a high humidity environment.

FIG. 15 illustrates different a textile sensor embedded in a garment in different locations.

FIG. 16 illustrates measured readings from sensors at different locations when a person conducts different activities.

FIG. 17 illustrates a sweating scale base on personal observations of wearer (3 people in each subfigure). The x-axis is the time from the moment they start to do exercise till they stop. The solid blue lines are the measured voltage readings for the sensor on the back. Blue dash lines are the measured moisture levels for the sensor on the armpit. The red line is the scale they feel of their shirt as listed in Table 2 below.

FIG. 18 illustrates the perceived sweat scale as a function of measured moisture level.

### DETAILED DESCRIPTION OF ILLUSTRATIVE EMBODIMENTS

The present disclosure may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable, and it should be understood that steps may be performed in any order.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range. In addition, the term “comprising” should be understood as having its standard, open-ended meaning, but also as encompassing “consisting” as well. For example, a device that comprises Part A and Part B may include parts in addition to Part A and Part B, but may also be formed only from Part A and Part B.

### 1. INTRODUCTION

Perspiration, or sweating, is a primary mean of thermal regulation for people. The amount of sweat is a significant indicator of one's physiological conditions [27]. Moreover, this information can be used to infer a person's comfort level, health condition, emotion, and exercise level [11, 27]. In some cases (e.g. babies), subjects do not have the ability to express their feelings and can lead to hypothermia, or even death [4]. Under such scenarios, well-placed temperature sensors can help detect body temperature elevation while an event is in progress. If one can sense sweating before subjects lose thermo-regulation ability, one can find out discomfort in time and thus prevent problems from getting worse [6, 27]. Furthermore, in non-medical situations, the amount of sweat can serve as a referential physical condition for personal body training [26] and an indication of hyperhidrosis [29]. Therefore, tracking the sweat level of a person provides important information for user comfort and health.

Prior work has explored approaches to detect sweating conditions. Electrical impedance and thermal conductivity have been adopted to measure the hydration of the skin [5, 9]. These pioneer researches are performed on subjects that are stationary, which can be impractical in a real-world scenario (e.g., exercises) [28]. Merilampi et al. proposed and demonstrated that it is possible to achieve binary measurements through scanning the frequency response of the passive UHF RFID textile tags [16]. Gao et al. utilized chemical sensors to detect and analyze the components of the sweat [7]. Their focus on sweat sensing is the component instead of the quantity or measurements that indicate the comfortableness of the user. Lie et al. designed a conductivity based sweat monitoring sensor that needs to be worn with a 3D printed mold [13]. Wei et al. presented a conformal sensor that measures the sweat rate and level through a sweat absorber and an inter-digitated capacitance sensor [28]. The sensor needs to be stuck to the skin, which can cause inaccurate reading or discomfort for sensitive and hairy skin. Therefore, it is a challenge to be able to sense people's sweat level through a non-intrusive way.

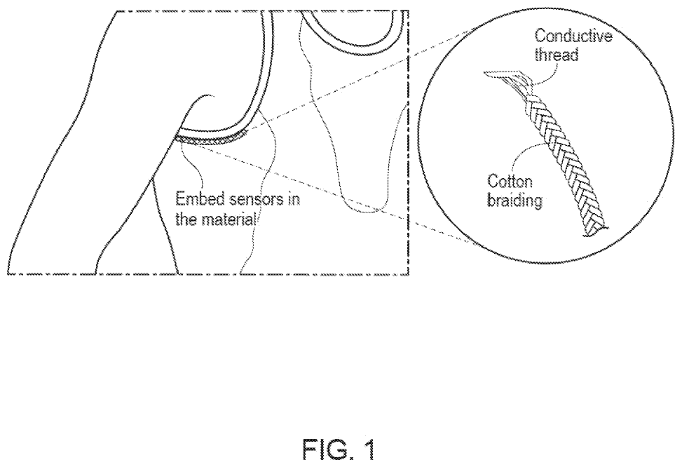

Provided here is a new sensor assembly that can measure an individual's sweat level and is robust against body movements. It can include conductive threads twined with insulating (e.g., cotton) braids. (Cotton is used as an illustrative material in this disclosure, but the present technology is not limited to cotton.) The concept includes measuring resistance between the conductive threads with a sensor, which changes subject to the amount of sweat between the cotton braids. However, weaving conductive threads directly into cotton may not guarantee a stable contact between cotton braids and conductive threads, especially when people move. In an aspect of the disclosed design, a 3D structured cotton cover is braided onto three conductive threads to maintain a stable structure, as shown in FIG. 1. The threads are then positioned to form a triangular structure that can keep liquid in between. Finally, another outer cover, such as a cotton cover, is braided to hold the structure in place. Such structure allows the sensor to be easily integrated to clothes of any material while still able to conduct sensing. When the sweat soaks the (cotton) cover, the resistance between the thread changes due to conductivity of the sweat.

The multiple-thread design allows stable sensor readings even when the user conducts strenuous activities. Additionally, the stability of the braiding structure makes the sensor washable since the materials are mainly cotton and conductive threads. The sensor is also low-cost and can be produced using techniques that are standard for machine weaving [25].

The sensor assembly comprises:

a 3D structured conductive thread based textile sensor that allows the robust measurement of sweat level despite user's motions;

characterization of the sensor under different moisture levels; experiments were conducted experiment on different sensing conditions (e.g., motion and bending) to show its sensing robustness to sensor deformation; and

evaluation of a sweat level sensing system that utilizes the designed conductive thread based textile sensor to measure the on-body sweat level.

### 2. BACKGROUND

Sweat sensing has been explored in different aspects that approximately fall into two categories: sweat level estimation and sweat component analysis. Sweat sensing is also closely related to moisture level sensing. In this section is introduced related work in these domains.

### 2.1 Sweat-Level Estimation

For sweat level estimation, various sensor designs have been explored. Electrical impedance and thermal conductivity have been adopted to measure the hydration of the skin [5, 9]. The pioneer research is done on users in a stationary condition [28], which may be impractical in a real-world scenario. Merilampi et al. proposed and demonstrated that it is possible to achieve binary measurements through scanning the frequency response of the passive UHF RFID textile tags [16]. Merilampi determines whether a person sweats instead of the amount or measurements that indicate the comfortableness of the user. Wei et al. presented a conformal sensor that measures the sweat rate and level through a sweat absorber and an inter-digitated capacitance sensor [28]. Lie et al. designed a conductivity based sweat monitoring sensor that needs to be worn with 3D printed molds [13]. The Wei sensors need to be directly mounted to people's skin, which may have inaccurate readings or cause discomfort for sensitive and hairy skins.

### 2.2 Sweat Component Analysis

Conventional systems have targeted different sweat components and have presented various sensing platforms and system to achieve their goals. Schazmann et al. and Matzeu et al. developed sweat sensing platforms for monitoring sodium in sweat [15, 23]. Rose et al. designed a patch sensor that can monitor the electrolytes in sweat [22]. Gao et al. utilized chemical sensors to detect and analyze the components of the sweat in real-time with wireless sensors. [7]. These works demonstrate the importance of the sweat sensing. However, each of these conventional systems fails to consider the level of sweat.

### 2.3 Moisture Sensing

Studies have been done on moisture sensing through the conductivity and resistance profiling. It has been applied for soil monitoring [2, 10, 21] as well as air humidity sensing [19, 20]. The general idea is to measure the resistance changes between two points on target that can hold/absorb water (e.g., soil). The disclosed sweat sensor utilized the mechanism that have been used to measure the water quantity in soil and air and modified it to detect conductivity of the sweat on cotton threads. The disclosed 3D structured cotton braiding can absorb the moisture and maintain a stable measuring condition to achieve sweat monitoring robust against human motion.

### 3. SWEAT PHYSIOLOGICAL BACKGROUND

The disclosed sensor targets at the measurement of the amount of sweat, which is an important indicator of human comfort level as well as physical conditions. In general, people have two types of sweat glands, the eccrine and apocrine sweat glands. Eccrine sweat glands excrete directly onto the surface of the skin while apocrine sweat glands do not [11, 27]. In fact, the clear secretion produced by eccrine sweat glands is termed sweat, which is the sensing target of this work. One of the major functions of eccrine sweat gland is the thermoregulation [30]. The amount of the sweat a person secreting indicates the thermal condition of the person—how comfortable they are—and then regulated by neural and hormonal mechanisms [6, 27]. Therefore, the sweat level is a good reflection of the comfortableness of individuals. In addition, the total volume of sweat produced depends on the number of functional glands and the size of the surface opening [8]. People who exercise more often and are more fit tend to have more sweat glands. Therefore, the sweat rate can be used as a reference of the personal fitness [26]. Sweat consists mostly of water. However, because it is derived from blood plasma, it also contains electrolytes, such as sodium, which makes the sweat conductive [30]. The conductivity of the sweat has been studied for various purposes. The measurements of the sweat conductivity can be used as the diagnostic test for cystic fibrosis [12, 14]. It has been used as an indicator of the sweat, as mentioned in other work [13]. These prior studies on the sweat conductivity indicate that when the sweat soaks the cotton, one can detect the sweat through resistance changes. However, their studies focused on the conductivity variation between subjects instead of the measurement of the sweat level.

### 4. OVERVIEW

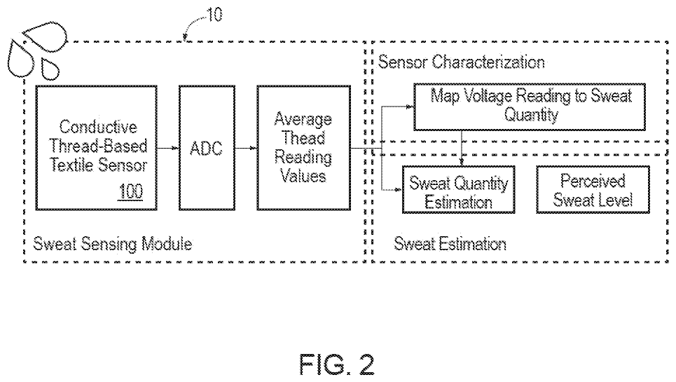

The disclosed sweat level monitoring system comprises three parts as shown in FIG. 2, including a sweat sensing module 10, the signal characterization, and the sweat level estimation. The sweat sensing module 10 comprises a conductive thread based textile sweat sensor 100 to detect the voltage value that indicates the moisture level between the conductive threads in the sensor 100. The output, in the form of divided voltage, is then converted to digital signal by the ADC on the module. Finally, the sensing module averages the ADC (analog-to-digital converter) readings corresponding to the two conductive threads in the sensor as the final measurement. The details of the design consideration are further discussed in Section 5 below. To map the measured sensor output (in voltage) to the quantity of the moisture between threads (e.g., in the unit of mg), sensor characterization is performed. During sensor characterization, known volumes of salt solution can be applied to the sensor and the voltage output of the sensor is measured. Then one can conduct a fitting based on a simplified equation of solution resistance, which is explained below. A goal of the system is to obtain a sweat level of the individual. Therefore, once one obtains the characteristics of the sensor, one can utilize that to convert the measured voltage to the quantity of the sweat detected by the sensor. In addition, for applications such as elderly care and exercise monitoring, directly informing the user of the sweat quantity in mg is not an intuitive way to describe their sweat condition, because each individual can have different sweat conditions. However, each person can also have their personal definition of sweat level based on their feelings. The system further conducts the perceived sweat level estimation based on the historical information provided by the user. Therefore, the disclosed sweat level estimation outputs the measured moisture level as well as the perceived sweat level based on the users' experience.

### 5. SENSOR DESIGN

To estimate the sweat level, a sensor can have different measurable attributes that change with the amount of the sweat the sensor will perceive. In addition, to non-intrusively sense the sweat level or people's perceived sensing, the sensor can contact for sample collection and measurement without being stuck to the skin directly.

One intuition is that, when people sweat at different amounts, their shirts show different amounts of soaking marks. Considering the human sweat contains electrolytes, different amounts of sweat mean different amounts of electrolytes, whose resistances can be measured. Based on a conductivity equation, this resistance has a relation to the amount of sweat between two conductive ends (e.g., the cross-sectional area). From a material perspective, the conductive threads can be woven into the cloth to achieve these considerations. However, the distance between the conductive ends also affects the resistance value. Therefore, a robust sensor structure is needed to maintain a consistent mapping relation between the amount of sweat and the measured resistance.

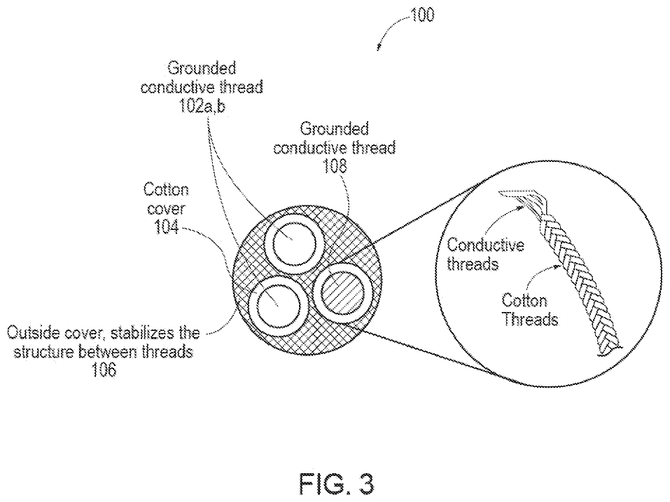

In an aspect, the sensor comprises multiple (e.g., three) conductive threads with braided cotton covers, which can absorb sweat for measurement purposes. To achieve a robust structure, each conductive thread is covered in a cotton braiding separately. Then the cotton-covered conductive threads are then covered by another cotton cover to keep the contacting surface between the threads stable and consistent. FIG. 3 illustrates a cross-sectional view of the sensor 100, which comprises conductive threads 102a,b, cotton covers 104 outside the conductive threads 102a,b, and a most outside cotton cover 106. The stability of the cotton cover 104, as well as the relative position between different cotton covers 104, is crucial for the sensor design.

### 5.1 Conductive Thread Design

To stably measure the sweat induced conductivity change between the threads 102a,b, one can place three threads in each sensor for at least the following reasons. First, from three conductive threads, two measurements can be obtained between the target thread and a grounded thread 108. One can take the average of these two measurements as the final measurement. This enhances the sensor's sensitivity for two reasons: 1) when the amount of the sweat is low, e.g., only one side of the sensor is directly contacting the sweat, averaging multiple threads allows the system to take multiple sweat-sensor contacting conditions into account and achieve a robust estimation of the sweat level, 2) when the human motion causes the thread to bend, which can affect the measurement, averaging multiple threads of different bending conditions can be more robust to the shape distortion caused sensing variations. Another reason to have three conductive threads is that three threads will be packed together with a triangle sectional view, and it is geometrically stable.

### 5.2 Cover Braiding

In order to measure the sweat, and to maintain constant separation between conductive threads, the cotton cover 104 can be braided on the surface of the conductive threads 102,a,b to absorb and hold the sweat in place. The cotton cover 104 is built from cotton threads following the method of square knots as shown in FIG. 3. One can choose square knots based on stability considerations, as shown in FIG. 3 on the right side. A purpose of a braided cotton cover is that it coats a conductive thread with an evenly distributed absorbing layer. Since cotton is one of the most absorbent materials, which is able to absorb up to 27×its weight in liquid [18], it gives a wider dynamic measuring range once applied to conductive threads. With the measure of the amount of liquid that cotton fibers are able to absorb, the range of measurable resistance can be determined. In addition, the resistance of cotton fibers is determined by the whole range of moisture contents (i.e., 1% to 22.4%): Log R=−9.3 log M+B, where R is the resistance of the sample in megohms, M is the moisture content in percent of the dry weight, and B is a constant. By rewriting the above equation one can get R=′M-^{9.3}, where B==Log B′. This behavior of cotton traces back to the fact that a decrease in the moisture of the cotton causes an increase in the general level of resistance [17].

### 5.3 Moisture Level to Voltage Conversion

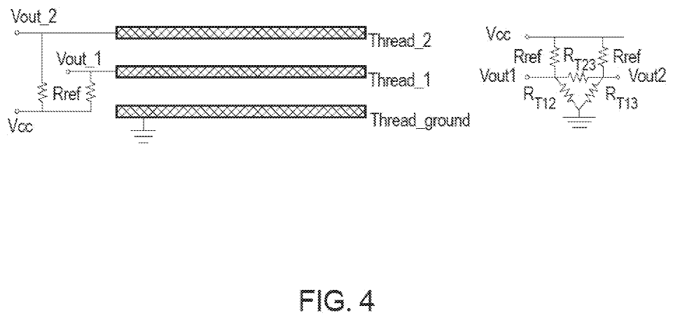

To convert the moisture level between a pair of conductive threads into the voltage based on Murphy, the cotton between the conductive threads can be considered a resistor R_{sweat}, whose value varies with the moisture level. When the cotton coat is completely dry, its resistance can be considered infinite. For each conductive thread pair, one of the threads connects to the ground. A reference level can be setup by setting the output to Vcc when the cotton is completely dry. Another known resistor R_{ref} is then used to partake the voltage so that when the cotton is dry (where one can consider R_{sweat}=∞) the electric potential between R_{sweat} and R_{ref} equals to Vcc. FIG. 4 shows the circuit design. The output of the circuit is calculated through the equation Vout=Vcc*R_{sweat}/(R_{sweat}+R_{ref}). An equivalent circuit is shown on the right hand side of FIG. 4, where R_{T12}, R_{T13}, and R_{T23} are equivalent resistors between thread pair Thread_1 and Thread_2, Thread_1 and Thread_3, and Thread_2 and Thread_3 respectively. Ideally, when the solution is distributed onto threads evenly, the value of R_{T12} and R_{T13} should be the same. It follows that the potential difference of Vₒᵤₜ₁ and Vₒᵤₜ₂ are the same and the resistance between Thread 1 and Thread 2 can be ignored.

### 6. SENSOR CHARACTERIZATION

To the best knowledge, this is the first work that utilizes a 3D structured cotton braiding with conductive threads to measure the amount of the sweat. To obtain a mapping between the sensor output and the on sensor sweat quantity, an experiment for sensor characterization is used.

### 6.1 Characterization Settings

A goal of the sensor characterization is to find the relation between the sweat quantity on the cotton threads and the voltage value of the sensor. Since the sensor measures the voltage value between conductive threads, it is important to maintain a stable sensing condition, including the temperature and humidity. To achieve that, the characterization experiment is preferably done in a room of consistent temperature and humidity.

A sodium chloride solution can be used to maintain the consistency of the solution conductivity in the characterization experiment. Prior research shows that the sodium concentration of human sweat can vary between 117 mEq per L to 172 mEq per L with an average of 137.8 mEq per L [3]. It indicates that the sweat conductivity from different people can vary, but within a range. Considering all the electrolytes in human's sweat, including sodium, potassium and all other components, the experiment used 1 percent sodium chloride solution to simulate the conductivity of the human sweat [24].

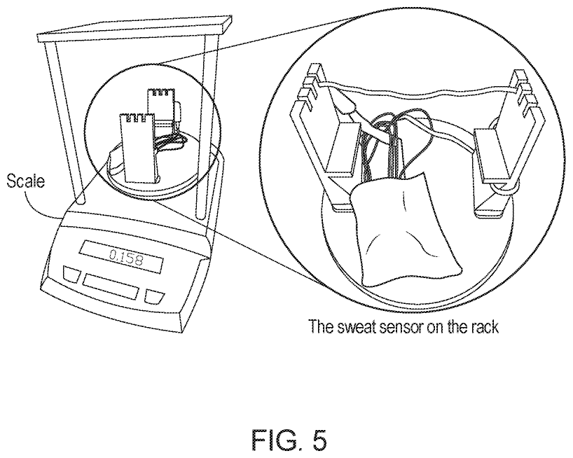

FIG. 5 shows the experiment setting. A 10 cm sensor is fixed on a 3D-printed rack, and the entire rack is placed on a scale that can measure up to 1 mg weight difference. For each trial, approximately 300 mg solution can be first applied on the thread to reach a maximum absorbance. Then after the solution evaporates to around 150 mg, data can be recorded. The waiting can take 10 to 15 minutes. One can record the sensor readings (voltage) and the quantity of the rest solution (mass) on the thread, during the process when the solution on sensor evaporates. These measurements are then used to generate the fitting function, which will be further discussed in Section 6.2.

### 6.2 Sensor Reading Mapping

The relationship between the target physical condition—the sweat quantity—and the measurable circuit changes are considered when using the sensor 100. When different amounts of solution are applied to the thread, it changes the resistance between the two conductive threads. This change in resistance leads to the change in the voltage output of the division circuit.

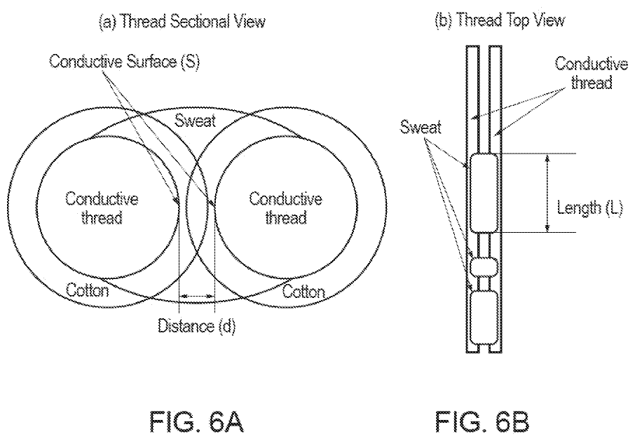

The sensor 100 can fit the simplified electrical conductivity model as follows. The resistance between the two conductive threads can be calculated as Rx=R·d/S, where, R stands for resistivity [1], d is the distance between the conductive threads, and S is the conductive thread surface that is soaked by sweat, which depends on the amount absorbed. FIG. 6 illustrates this simplified model with the sectional and top views of a pair of conductive threads. The minimum distance between two conductive thread surfaces can be used as a representative value of the distance d between the conductive threads. Due to the stable braiding structure, the distance d can be considered a constant. On the other hand, when the sweat soaks on part of the sensor threads, as shown on the right-hand side of FIG. 6 (i.e., FIG. 6B), the conductive thread surface S is proportional to a length L of the soaking area. Therefore, when more sweat is absorbed by the cotton cover, the effective conductive thread surface S is larger. The disclosed sensor characterization utilizes this relation as the fitting function to estimate the mapping between the voltage readings and the amount of the solution absorbed by cotton threads.

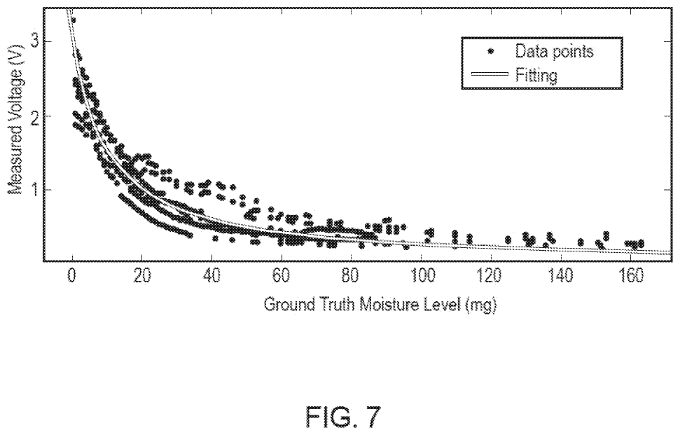

FIG. 7 shows the measurement of the resistance between the measured conductive thread and the ground thread. In an aspect, data is collected for eight different sections of the sensor braid, which can be, for example, 10 cm long in a room where the humidity and temperature are controlled and constant. For a solution quantity of less than 80 mg, the threads show a consistent decreasing trend in resistance. However, most of the resistance variations are reflected by the solution quantity change from 0 mg to 30 mg, where the difference in voltage is from 3.3 V to 0.77 V. Different threads also show higher variation compared to the trend for solution quantity between 30 mg and 80 mg. A function that can fit these readings, for example, is p₁/(x+p₂), which is based on the resistance function discussed in Section 3.

It is important to understand the sensibility of the sensor regarding the dynamic sensing range as well as sensitivity so that users can interpret the sensor readings (in voltage) to the sweat quantity. Based on the experimental observation, when the moisture level is beyond 80 mg, the voltage readings fluctuate at 0.35 volt. Therefore, the dynamic sensing range of the sensor is approximately up to 80 mg. Since the measured voltage and the moisture level is not linear, the sensitivity of the sensor can be analyzed in different ranges. The sensitivity in these ranges can be summarized based on the fitting curve obtained from FIG. 7. Table 1 shows the target sensing range in the unit of mg, which corresponds to the x-axis in FIG. 7. For each sensing range listed, a sample point can be selected and the derivative of the fitting curve at that point can be obtained. The derivative of that sample point can be considered as the sensitivity of the target range.

TABLE 1
Sensor Sensitivity

| Target Range (mg) | 0-1 | 1-10 | 10-20 | 20-40 | 40-80 |
|---|---|---|---|---|---|
| Sample Point (mg) | 0.5 | 5 | 15 | 30 | 60 |
| Sensitivity (V/mg) | 0.2856 | 0.1392 | 0.0781 | 0.0194 | 0.0063 |

### 7. SWEAT ESTIMATION

The system can estimate at least two types of sweat level information: the measured moisture level of the shirt and the perceived sweat scales. The measured moisture level is an absolute quantity value mapped based on the sensor readings. However, when used in real-life applications, it is difficult for people to understand the absolute value of their sweat quantity in mg. To allow intuitive understanding of the sweat condition, the perceived sweat scales can be defined to describe different sweat conditions.

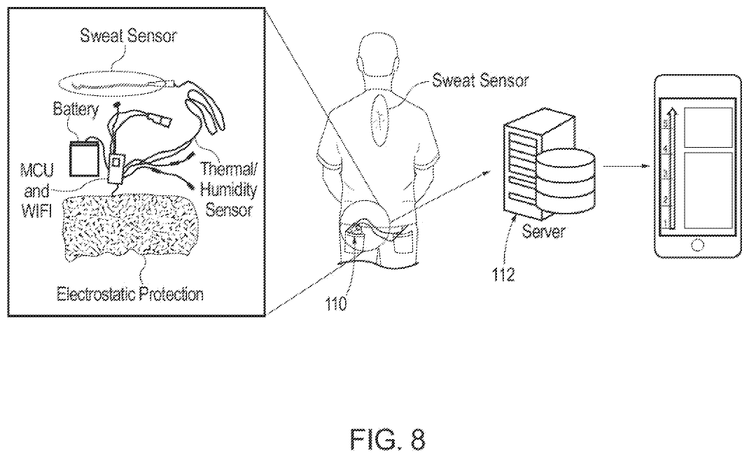

The sensor 100 can be used to detect the quantity of the sweat absorbed by the cotton threads on the sensor 100. Once the sensing module 110 measures the sensor resistance change in the form of divided voltage changes, it sends the measurements to a server 112 (e.g. processor) as shown in FIG. 8. For each sweat sensor 100 with three conductive threads, two readings can be obtained from the non-grounded conductive threads 102a,b and the average voltage value V_{out_avg} can be obtained. Then the system maps the voltage reading to the sweat volume based on the mapping relation learned in the sensor characterization as discussed in Section 6.

The server 112 calculates the moisture level in mg and estimates each user's perceived sweat level based on historical data. The user can check the measured sweat level as well as their estimated perceived sweat level on a mobile app. Based on the measured moisture and the person's labeling, the system can further estimate the perceived sweat scales as an objective standard of sweat. Note that this is an objective measurement. Therefore, individuals need to provide labeling to obtain their personalized perceived sweat level estimation.

### 8. EVALUATION

To evaluate the disclosed sweat sensing system, two conductive thread-based textile sweat sensors were built, each 10 cm with three threads in each sensor (Section 8.1). To demonstrate the robustness of the sensor under different sensing conditions, such as motion causing deforming and bending, a series of experiments can be conducted targeting at different sensing variables through controlled experiments (Section 8.2). Finally, the system can be evaluated through a real-world human experiment with multiple participants under different sweat conditions (Section 8.3).

### 8.1 Implementation

A conductive cover can be used as to electrically isolate a sensing board, as shown in FIG. 5. The sweat sensor 100 is installed on a rack and the board is covered by the blue conductive cover.

In addition, because the working principle of the sensor is to measure the changing resistance, the ambient conductive materials that are in a similar resistance range can affect the sensing accuracy. The reference resistance R_{ref} that can used is, for example, 1M Ohm.

### 8.2 Sensing Condition Characterization

For sensing people's sweat level, the disclosed sensors can be embedded into people's clothes. Therefore, a robust sensor performance under practical sensing conditions is important for the sensor design.

When people wear the cloth with the embedded sensor, their motion may cause the sensor 100 to continuously deform. The disclosed braided 3D structure of the sensor 100 makes the electric condition between embedded conductive threads stable, hence making it robust sensor deformation caused by movement. The following sections show the robustness of the sensor 100 under different motion velocity and bending angles.

Further, different people may have different sweat concentration as discussed. When the sensor is used for a long duration, the sweat may be accumulated on the sensor causing a high concentration of electrolytes.

### 8.2.1 Condition Characterization Procedure

Experiments were conducted where the initial amount of solution is applied to the sensor 100 until the cotton on the sensor saturates (the resistance value does not drop even more solution is applied). The sensor reading curves are measured and compared during the solution evaporation to demonstrate sensing consistency. To do that, a 1% solution was applied to the sensor until it is fully absorbed. Then the sensor readings were measured while the solution is evaporating from the sensor under different motion conditions. For investigating similar parameters, environmental factors, such as room temperature and humidity, were kept the same to make the evaporation process similar.

### 8.2.2 Motion-induced Sensor Deformation

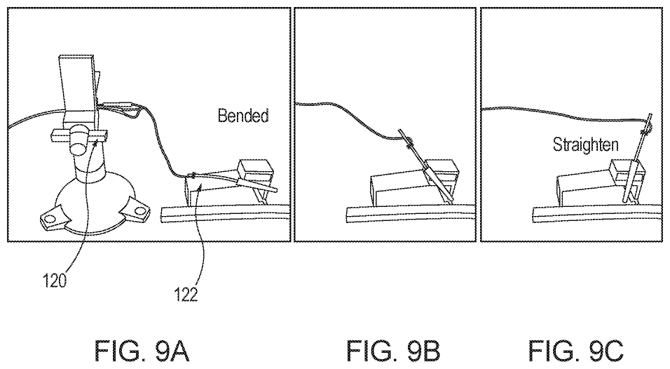

Sensor motion experiments were conducted with the setup shown in FIGS. 9A-9C. To create consistent and comparable motion conditions, one can fix one end of the sensor by a board holder 120 as shown on the left hand of the setup, while fixing the other end of the sensor 100 to a plastic arm 122 that is connected to a servo motor. The servo motor is programmed to rotate between 0. (FIG. 9A) and 90° (FIG. 9C), which correspond to the sensor conditions of bend and straighten, respectively. One can consider the motion from position 0° to position 90° and then back to 0° as a round. The three motion conditions investigated were 1) completely static, 2) slow motion—10 seconds per round, and 3) fast motion—2.5 seconds per round.

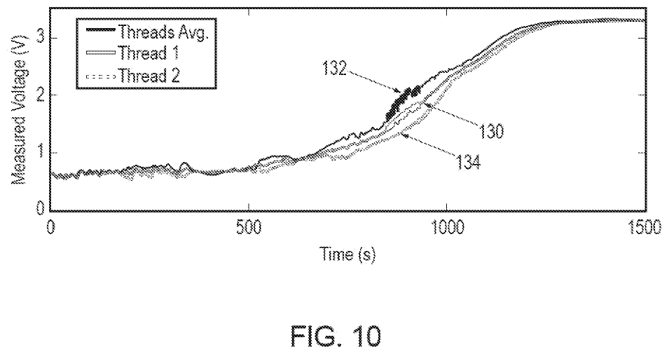

FIG. 10 illustrates readings from the two threads when moved in slow motion. The blue line (130) is the average reading from the two threads while the red and yellow lines (132 and 134) are the readings from Thread 1 and Thread 2, respectively. There is slight noise on Thread 1 between 900 and 1000 seconds and there is relatively low noise on Thread 2 during that period. This could be caused by the different effects of bending direction and position on different threads. Therefore, by averaging the values from the two readings from the two internal threads, one can observe a stable curve that is robust to the motion.

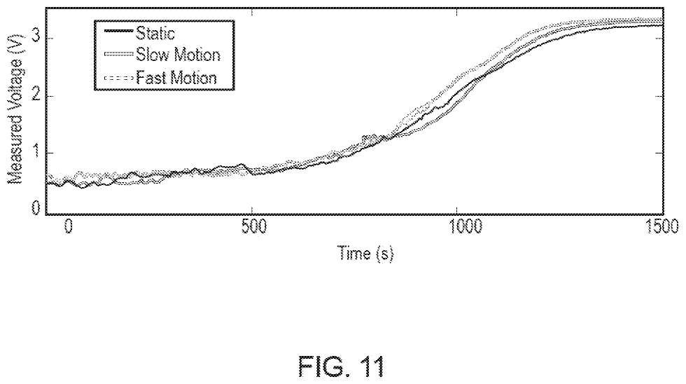

FIG. 11 illustrates a graph of the three motion conditions, which further illustrates the robustness of the sensor 100. Despite the different motion conditions, the system shows consistent reading curves when the water evaporates from the sensor. Accordingly, the disclosed sensor is demonstrably robust to motion behavior.

### 8.2.3 Sharp Bending at Different Angles

When the sensor is woven in the clothes, it may be bent at a sharp angle, which may cause a significant change in the distance between the conductive threads and the pressure on the cotton threads. To characterize the partial sharp bending and its effects on sensor reading, experiments were done where the sensors are bent intentionally to a specific degree, including 30, 60, and 90 degrees.

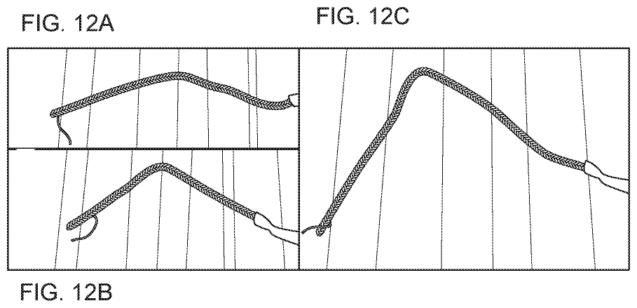

FIGS. 12A-12C demonstrates the different bending angles of the sensor.

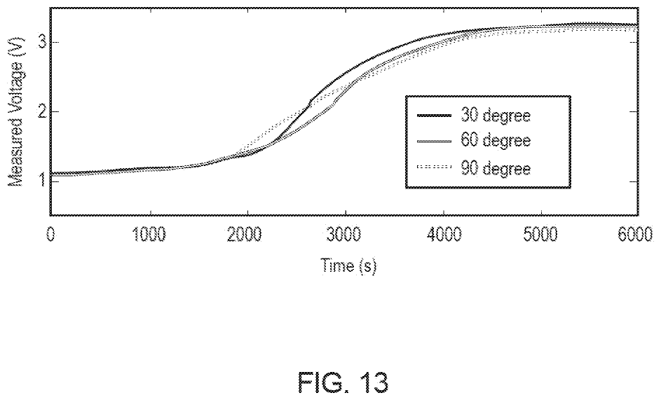

FIG. 13 shows the measured voltage change when the sensor is bent at different angles. During the evaporation progress, the measured voltage change for different bending degrees is consistent. This shows that the bending angle investigated did not affect the sensor reading, and the disclosed braided 3D structure is robust to deformation as sharp as shown in this experiment.

### 8.2.4 Variation in Solution Concentration

When different people have different sweat concentration, or the accumulative sweating changes the sweat concentration on sensor, the high concentration of the electrolytes may alter the sensibility of the sensor. To investigate the effects of difference concentration level of the sweat, the characterization experiment was conducted with four different level of solution, which are tap water, 1%, 2%, and 4% salt in tap water respectively. Note that after each experiment the sensor was washed to ensure no electrolytes remained on the sensor.

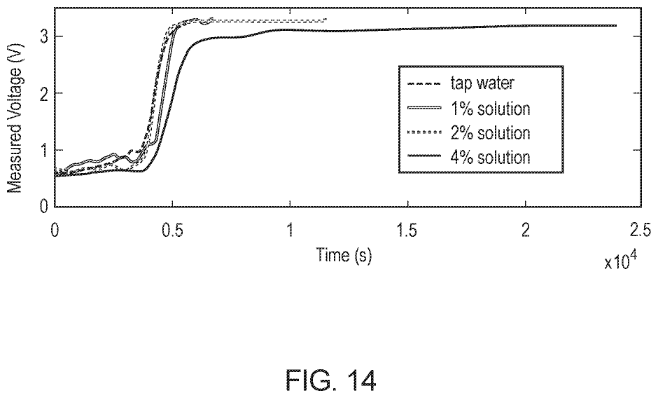

The measured voltage curves under these concentration levels during the evaporation process are shown in FIG. 14. As discussed before, one can consider 1% solution a similar electrolyte concentration as human sweat [24]. One can observe that for the solution that is significantly higher than sweat—4% solution—the measured voltage does not goes back to 3.3 volt after it dries. This may be caused by the high concentration of the electrolytes that is conductive when the humidity level in air is high. The low concentration solution investigated demonstrated a consistent performance when the solution evaporates. This indicates that the sensor's sensitivity and dynamic range is consistent for normal people's sweat concentration range (the variation is less than 2× of the average concentration) [3, 24].

### 8.3 On-Body Experiments

To evaluate the disclosed sweat sensing system under a realistic scenario, an experiment was performed on people, where sweat sensors were sewn at different locations on a shirt. As discussed below, sweat sensing at different body parts under different sports levels vary for different people.

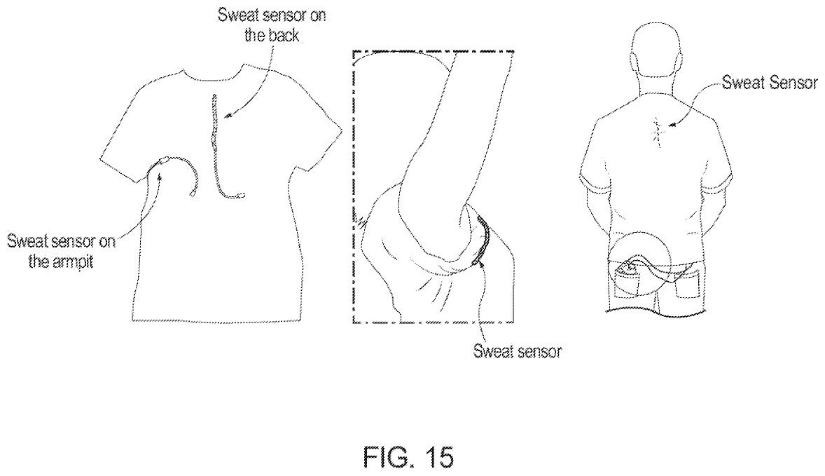

As discussed in Section 6, the system outputs two types of sweat level information: the measured moisture level and the perceived sweat scales. Multiple trials were performed of exercise experiments on participants and the historical perceived sweat scale was used to build a prediction model. The prediction model was applied to the testing data and used to calculate the prediction error to evaluate the system performance. To investigate the sweating behavior on humans, two sensors (10 cm each) were placed at the back and the armpit of the shirt, as shown in FIG. 15. The armpit and the back produce a lot of sweat because there is a relatively high density of sweat glands at those locations.

### 8.3.1 System Parameters

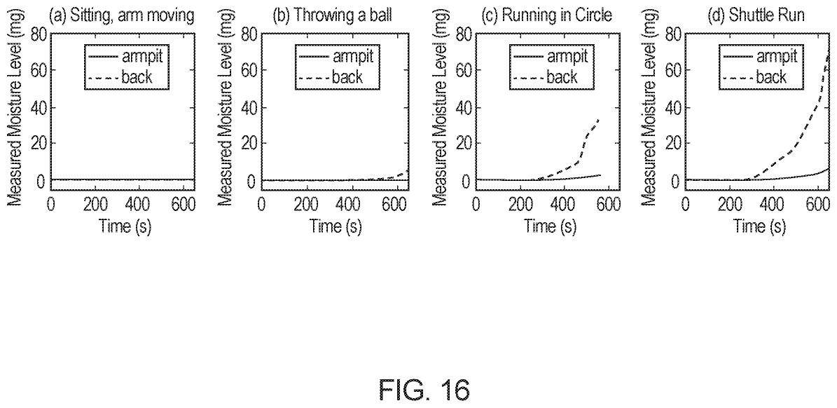

To demonstrate the performance and robustness of the disclosed system, experiments were conducted where the participants are doing activities with different sweating profiles, including moving arms while sitting, throwing a ball at a wall, running in circle and shuttle run. The sensing results are shown in FIG. 16, where blue solid lines are the sensing values from the sensor sewed at the armpit and red dash lines are the sensing values from the sensor sewed on the back.

Activity Comparison

The four activities selected are representatives of four different activity levels. Therefore, the system detects different sweating profiles for each. The arm moving is the mildest activity. Therefore, after 650 seconds, readings from both sensors are still around 0 mg as shown in FIG. 16 (a). On the other hand, when the participant throws a ball, the activity level is higher, which causes the back and armpit sensor readings to start to increase after 500 seconds of activity as shown in FIG. 16 (b).

For more intense activity (e.g., running), the sensor reading starts to increase even earlier—around 300 seconds after the participant started the activity—as shown in FIGS. 16 (c) and (d). However, for the two different types of running, even though the participant experiences different activity intensity, the measured sweat sensor readings showed similar change trends and rates. This indicates that the intensity of the exercise is not correlated to the sweating rate. The duration of the exercise affects the sweat rate and quantity more than intensity.

Body Part Comparison

Two sensors were placed at two high sweat locations on people. For the same participant who conducted different activities, the back always produced more sweat than the armpit. For the arm moving activity, both body parts did not show clear sweat, as shown in FIG. 16 (a), due to the non-intensity of the activity. For the ball throwing activity, the measured moisture level is shown in FIG. 16 (b), the back sensor detects more sweat than that on the back after 500 seconds.

Similarly, for the rest of the activities that are more intense than the ball throwing, as shown in FIGS. 16 (c) and (d), the sensor on the back showed a faster-increasing trend than that of the armpit. This could be caused by the motion of opening armpit allows faster sweat evaporation rate compared to the sweat on the back.

### 8.3.2 Perceived Sweat Scale

An individual may perceive the sweat level differently based on their comfort sensation. One can further evaluate how the measured moisture level can assist the estimation of perceived sweat scale of people.

One can consider five different levels of perceived sweat scale and summarize it in Table 2. Running was selected as the activity to test, and each participant runs multiple trials in a room with a stable temperature. During the experiment, participants were encouraged to keep themselves hydrated. Participants were also asked to answer the question ‘how wet do you think your shirt is?’, and answered on a scale from 1 to 5, where 1 is dry, and 5 is soaking wet. Discussed next is the perceived sweat scale profile from three different participants and also how to predict individual perceived sweat scales based on personal historical sweat sensing data.

TABLE 2
Perceived sweat scale

| Scale | 1 | 2 | 3 | 4 | 5 |
|---|---|---|---|---|---|
| Description | dry | start to sweat | light sweat | heavy sweat | soaking wet |

Person Comparison

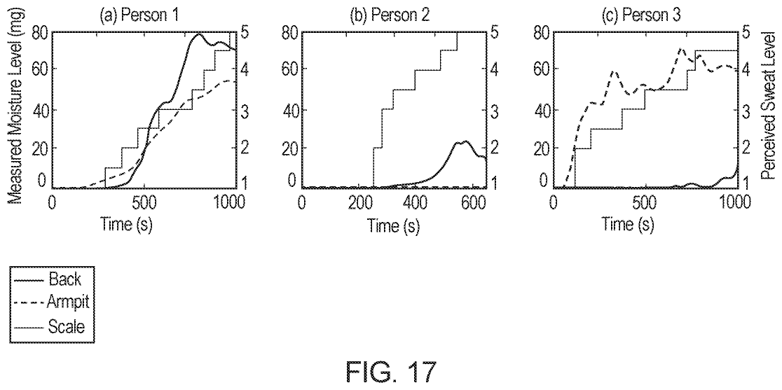

FIG. 17 shows examples of three participants marking perceived sweat scale (as listed in Table 2) and the corresponding sensor measurements. The sensor readings between the ‘start to sweat’ and the ‘heavy sweat’ level are the most sensitive range. This perceived sensing range corresponds to the measured moisture level below 60 mg in general, which is the sensitivity of the sensor as discussed in Table 1.

In addition, different people may have different sweat profiles. For example, person 1 and person 3 have heavy armpit sweat, while person 2 sweats on the back faster and has a lower tolerance to sweat on the shirt. The perceived moisture level of person 1 and person 3 has a stronger correlation to the measured moisture level at armpit than that of person 2. Person 3 gets sweaty quickly, and her armpit sweat shows a significant increase in the first 100 seconds. However, the back sweat profile only goes up to around 15 mg during the entire exercise. This may be due to the sports bra that the participant wore absorbed most of the sweat and made the absorption of the sweat on the sensor more difficult and uneven. Since the disclosed sensor can be integrated into clothes, for female users, the sensor can also be integrated to the edge of the bra.

Scale Prediction

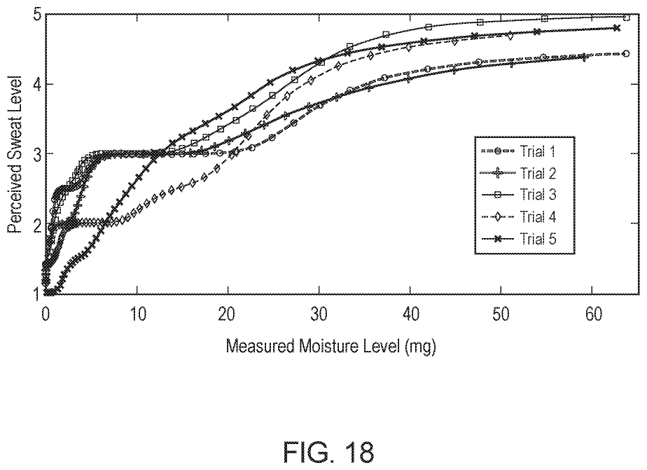

Further trials were conducted on the same person and collected the measured moisture level together with their perceived sweat scale. For each trial, kernel smooth regression was applied between the measured moisture level and the recorded perceived sweat scale. FIG. 18 shows the regression of five different trials from one participant. Despite the fact that the trials show different characteristics, the overall trends are similar and the perceived sweat level difference for the same measured moisture level is mainly within 1 level. Therefore, one can further utilize one trial as the testing data and the rest as the training data to conduct prediction based on the kernel smooth regression model built upon the training data. Cross-validation was done, and the average perceived sweat scale estimation error was approximately 0.4 with a standard deviation of approximately 0.28.

### 9. DISCUSSION

This section focuses on the cotton thread structure and how it may affect the sensor sensibility. The technology can be applied in a large-scale deployment to obtain environmental information from individual physical responses.

### 9.1 Sensibility and Sensor Structure

The 3D braiding structure introduced in Section 5.2 was used to achieve stable sensing on the sweat level. In the disclosed sensor characterization experiment, as more sweat is absorbed, the sensitivity of the sensor decreases. Also, based on the observation, one can define (without limitation) the dynamic sensing range of the sensor as between 0 mg to 80 mg. Based on the principle of this work, the 3D braiding structure, as well as its material, can affect the sensitivity and the dynamic sensing range of the sensor.

Cotton thread thickness is one structural parameter that may affect the sensitivity of the sensor. If a relatively thick cotton thread is used, the distances between conductive threads will increase such that the resistance is higher when the same amount of sweat is applied to it, and the sensor will have relatively less sensibility to the low moisture level but higher sensibility to the high moisture level. When that happens, the dynamic sensing range increases. In that case, the sensitivity of the sensor will decrease, because the V/mg can be measured decreased with the increase of the dynamic sensing range. If the sensor length increases, the sensitivity of the sensor does not change. A longer sensor has a larger sensing surface, and it covers a larger skin surface and obtains more sweat. One may consider it has a higher sensibility when the sweat level is low.

In these illustrative experiments, cotton thread was selected to braid the 3D structure as the absorber for sweat; if a material such as nylon is used, the evaporation of the sweat is higher. To use a different material, the evaporation rate can be used to calibrate the sensitivity model as well as the dynamic range.

### 9.2 Sweat Level Crowd-Sourcing for Environment Condition Inference

The disclosed technology may also be used for, e.g., applications such as monitoring subjects' hypothermia condition or exercise monitoring. The sensor can be used to predict the individual perceived sweat scale based on the measured moisture level. When the sensor is applied in a large-scale deployment (such as on patrons in a gym or a sports team), the crowd-sourced sensing data can be further used to infer the group condition, such as if a specific area is too warm or cold based on sensed human thermal regulation.

### 10. SUMMARY

The disclosed technology provides a stable yet wearable sweat sensor, and the present disclosure provides a conductive thread-based textile sensor that can be integrated into clothing for continuous perspiration sensing. The sensor can comprise three cotton-covered conductive threads and measures the moisture level between the conductive threads to infer sweat quantity. Each cotton-covered conductive thread can be, e.g., braided with a 3D structure, which allowed robust moisture level measurement despite the sensor deformation caused by human motions.

Sensor characterization was performed in connection with the mapping between the measured moisture level and the sensor voltage output. To evaluate the sensor and the system, sensing condition characterization was performed to demonstrate its robustness against the sensor distortion due to motion, bending, and solution concentration. Experiments with the prototype were also conducted with multiple volunteers to explore people's sweat profile. Also investigated was predicting people's perceived sweat scale based on the sensing data and, in one non-limiting experiment, achieved an average error of 0.4 levels when compared to a 5-level scale of human perception.

Features of the disclosed embodiments can be combined and/or rearranged within the scope of the disclosure to produce additional embodiments. The described embodiments are illustrative and non-limiting.

### Exemplary Embodiments

The following embodiments are exemplary only and do not serve to limit the scope of the present disclosure or the appended claims.

Embodiment 1. A sensor, comprising: a first textile assembly having a first conductive element and a first outer sheath that surrounds the first conductive element, the first outer sheath formed from a first non-conductive material that is configured to transport moisture through the first outer sheath; and a second textile assembly having a second conductive element and a second outer sheath that surrounds the second conductive element, the second outer sheath formed from a second non-conductive material that is configured to transport moisture through the second outer sheath, the first and second outer sheaths maintaining a separation between the first conductive element and the second conductive element along a length of the sensor.

Embodiment 2. The sensor of Embodiment 1, further comprising an outer cover that at least partially encases the first and second textile assemblies.

Embodiment 3. The sensor of Embodiment 2, wherein the outer cover is a knitted structure, a woven structure, a braided structure, a knotted structure, or any combination thereof.

Embodiment 4. The sensor of Embodiment 1, wherein the first non-conductive material and the second non-conductive material comprise at least one of a knitted structure, a woven structure, a braided structure, and a knotted structure.

Embodiment 5. The sensor of Embodiment 1, wherein the first outer sheath is in contact with the second outer sheath.

Embodiment 6. The sensor of Embodiment 1, wherein the first non-conductive material and the second non-conductive material comprise absorbent fibers.

Embodiment 7. The sensor of Embodiment 1, wherein the first non-conductive material and the second non-conductive material comprise moisture-transporting fibers.

Embodiment 8. The sensor of Embodiment 1, wherein the first non-conductive material and the second non-conductive material comprise cotton fibers.

Embodiment 9. The sensor of Embodiment 1, further comprising: a third textile assembly having a third conductive element and a third outer sheath that surrounds the third conductive element, the third outer sheath formed from a third non-conductive material that is configured to transport moisture through the third outer sheath, the third conductive element being grounded, the third outer sheath maintaining a separation between the third conductive element and the first and second conductive elements along the length of the sensor.

Embodiment 10. The sensor of Embodiment 8, further comprising an outer cover that at least partially encases the first, second, and third textile assemblies.

Embodiment 11. A sweat level monitoring system comprising: a sensor comprising: a first textile assembly having a first conductive element and a first outer sheath that surrounds the first conductive element, the first outer sheath formed from a first non-conductive material, and a second textile assembly having a second conductive element and a second outer sheath that surrounds the second conductive element, the second outer sheath formed from a second non-conductive material, the first and second sheaths maintaining separation between the first conductive element and the second conductive element, the sensor configured to detect a voltage value between the first conductive element and the second conductive element; and a processor configured to: receive the detected voltage value from the sensor, and map the detected voltage value to a moisture characteristic (e.g., a moisture level, an amount of electrolyte).

Embodiment 12. The sweat level monitoring system of Embodiment 11, further comprising: a memory configured to: store a table that includes a plurality of voltage values and a plurality of moisture characteristics, wherein each of the plurality of voltage values is associated with a respective one of the plurality of moisture characteristics, and wherein the moisture characteristic to which the detected voltage is mapped is one of the plurality of moisture characteristics.

Embodiment 13. The sweat level monitoring system of Embodiment 12, wherein the table further includes a plurality of perceived moisture values, wherein each of the plurality of perceived moisture values is associated with a respective one of the plurality of moisture characteristics and a respective one of the plurality of voltage values.

Embodiment 14. The sweat level monitoring system of Embodiment 13, further comprising: a human-machine-interface (HMI) configured to receive a perceived moisture value input by a user, the input perceived moisture value being associated with the detected voltage, wherein the processor is further configured to: compare the input perceived moisture value to a respective one of the plurality of perceived moisture values to which the detected voltage is mapped, and provide an indication to the user, via the HMI, if the input perceived moisture value is different from the respective one of the plurality of perceived moisture values.

Embodiment 15. The sweat level monitoring system of Embodiment 14, wherein the plurality of perceived moisture values is input into the table via the HMI.

Embodiment 16. A method for determining a moisture characteristic with a moisture characteristic monitoring system, the method comprising: detecting, via a sensor, a voltage value, wherein the sensor comprises a first textile assembly and a second textile assembly, the first textile assembly having a first conductive element and a first outer sheath that surrounds the first conductive element, the first outer sheath formed from a first non-conductive material, and the second textile assembly having a second conductive element and a second outer sheath that surrounds the second conductive element, the second outer sheath formed from a second non-conductive material, wherein the detected voltage value is detected between the first conductive element and the second conductive element; and determining the moisture characteristic based on the detected voltage value.

Embodiment 17. The method of Embodiment 16, wherein determining the moisture characteristic includes mapping the detected voltage value to the moisture characteristic stored in a table in a memory of the monitoring system, the table including a plurality of voltage values and a plurality of moisture values, wherein each of the plurality of voltage values is associated with a respective one of the plurality of moisture characteristics.

Embodiment 18. The method of Embodiment 17, wherein the moisture characteristic is a first moisture characteristic and wherein the voltage value is a first voltage value, the method further comprising: detecting, via the sensor, a second voltage value; measuring a second moisture characteristic that corresponds to the detected second voltage value; and storing, in the table in the memory, the second moisture characteristic associated with the second voltage value.

Embodiment 19. The method of Embodiment 18, wherein the first voltage value is detected at a first time, and wherein the second voltage value is detected at a second time, the first time being different from the second time.

Embodiment 20. The method of Embodiment 17, wherein the table further includes a plurality of perceived moisture values, wherein each of the plurality of perceived moisture values is associated with a respective one of the plurality of moisture characteristics and a respective one of the plurality of voltage values, the method further comprising: receiving a perceived moisture value; comparing the input perceived moisture value to a respective one of the plurality of perceived moisture values to which the detected voltage is mapped; and indicating if the input perceived moisture value is different from the respective one of the plurality of perceived moisture values.

Embodiment 21. The method of Embodiment 16, further comprising: determining a resistance (Rₓ) between the first conductive element and the second conductive element based on the detected voltage value, wherein the first conductive element is spaced apart from the second conductive element by a distance (d), wherein determining the moisture characteristic includes determining a surface area (S) of moisture on the first and second conductive elements based on a relationship defined by: Rₓ=R·d/S, wherein R is the resistivity of the first and second conductive elements.

Embodiment 22. A method, comprising: with a first textile assembly having a first conductive element and a first outer sheath that surrounds the first conductive element, the first outer sheath formed from a first non-conductive material that is configured to transport moisture through the first outer sheath; and a second textile assembly having a second conductive element and a second outer sheath that surrounds the second conductive element, the second outer sheath formed from a second non-conductive material that is configured to transport moisture through the second outer sheath, the first and second outer sheaths maintaining a separation between the first conductive element and the second conductive element along a length of the sensor, detecting a voltage related to an amount of moisture that places the first conductive element into electrical communication with the second conductive element.

Embodiment 23. The method of Embodiment 22, further comprising mapping the voltage to a moisture characteristic stored in a table, the table including a plurality of voltage values and a plurality of moisture characteristics, wherein each of the plurality of voltage values is associated with a respective one of the plurality of moisture characteristics.

Embodiment 24. The method of Embodiment 23, wherein the moisture characteristic comprises a volume, an accumulation, a rate of accumulation, a level of electrolyte, an evaporation, a rate of evaporation.

### REFERENCES

[1] Standard Test Methods for Electrical Conductivity and Resistivity of Water. Standard, ASTM International, PA, USA, 2005.
[2] A. Binley, P. Winship, L. J. West, M. Pokar, and R. Middleton. Seasonal variation of moisture content in unsaturated sandstone inferred from borehole radar and resistivity profiles. Journal of Hydrology, 267(3-4):160-172, 2002.
[3] G. W. Cage and R. L. Dobson. Sodium secretion and reabsorption in the human eccrine sweat gland. The Journal of clinical investigation, 44(7):1270-1276, 1965.
[4] P. Fleming, M. Levine, Y. Azaz, R. Wigfield, and A. Stewart. Interactions between thermoregulation and the control of respiration in infants: possible relationship to sudden infant death. Acta Paediatrica, 82(s390):57-59, 1993.
[5] T. Frodin, P. Helander, L. Molin, and M. Skogh. Hydration of human stratum corneum studied in vivo by optothermal infrared spectrometry, electrical capacitance measurement, and evaporimetry. Acta dermato-venereologica, 68(6):461-467, 1988.
[6] A. P. Gagge, J. Stolwijk, and J. Hardy. Comfort and thermal sensations and associated physiological responses at various ambient temperatures. Environmental research, 1(1):1-20, 1967.
[7] W. Gao, H. Y. Nyein, Z. Shahpar, L.-C. Tai, E. Wu, M. Bariya, H. Ota, H. M. Fahad, K. Chen, and A. Javey. Wearable sweat biosensors. In Electron Devices Meeting (IEDM), 2016 IEEE International, pages 6-6. IEEE, 2016.
[8] C. P. Hickman, L. S. Roberts, A. Larson, W. C. Ober, and C. Garrison. Integrated principles of zoology, volume 15. McGraw-Hill New York, 2001.
[9] X. Huang, W.-H. Yeo, Y. Liu, and J. A. Rogers. Epidermal differential impedance sensor for conformal skin hydration monitoring. Biointerphases, 7(1-4):52, 2012.
[10] C. K. Johnson, J. W. Doran, H. R. Duke, B. J. Wienhold, K. M. Eskridge, and J. F. Shanahan. Field-scale electrical conductivity mapping for delineating soil condition. Soil Science Society of America Journal, 65(6):1829-1837, 2001.
[11] K. Kurosumi, S. Shibasaki, and T. Ito. Cytology of the secretion in mammalian sweat glands. In International review of cytology, volume 87, pages 253-329. Elsevier, 1984.
[12] T. S. Licht, M. Stern, and H. Shwachman. Measurement of the electrical conductivity of sweat: Its application to the study of cystic fibrosis of the pancreas. Clinical chemistry, 3(1):37-48, 1957.
[13] G. Liu, C. Ho, N. Slappey, Z. Zhou, S. Snelgrove, M. Brown, A. Grabinski, X. Guo, Y. Chen, K. Miller, et al. A wearable conductivity sensor for wireless real-time sweat monitoring. Sensors and Actuators B: Chemical, 227:35-42, 2016.
[14] A. C. V. Mattar, C. Leone, J. C. Rodrigues, and F. V. Adde. Sweat conductivity: An accurate diagnostic test for cystic fibrosis? Journal of Cystic Fibrosis, 13(5):528-533, 2014.
[15] G. Matzeu, C. O'Quigley, E. McNamara, C. Zuliani, C. Fay, T. Glennon, and D. Diamond. An integrated sensing and wireless communications platform for sensing sodium in sweat. Analytical Methods, 8(1):64-71, 2016.
[16] S. Merilampi, H. He, L. Sydanheimo, L. Ukkonen, and J. Virkki. The possibilities of passive uhf rfid textile tags as comfortable wearable sweat rate sensors. In Progress in Electromagnetic Research Symposium (PIERS), pages 3984-3987. IEEE, 2016.
[17] E. Murphy and A. Walker. Electrical conduction in textiles. i the dependence of the resistivity* of cotton, silk and wool on relative humidity and moisture content. The Journal of Physical Chemistry, 32(12):1761-1786, 1928.
[18] K. Norton. Why Is Cotton Absorbent?, 2018. https://sciencing.com/cotton-absorbent-6662538.html.
[19] F. Opekar and D. Svozil. Electric resistance in a Nafion® membrane exposed to air after a step change in the relative humidity. Journal of Electroanalytical Chemistry, 385(2):269-271, 1995.

What is claimed:

1. A sensor, comprising:
a first textile assembly having a first conductive element and a first outer sheath that surrounds the first conductive element, the first outer sheath formed from a first non-conductive material that is configured to transport moisture through the first outer sheath; and
a second textile assembly having a second conductive element and a second outer sheath that surrounds the second conductive element, the second outer sheath formed from a second non-conductive material that is configured to transport moisture through the second outer sheath,
the first and second outer sheaths maintaining a separation between the first conductive element and the second conductive element along a length of the sensor.

2. The sensor of claim 1, further comprising an outer cover that at least partially encases the first and second textile assemblies.

3. The sensor of claim 2, wherein the outer cover is a knitted structure, a woven structure, a braided structure, a knotted structure, or any combination thereof.

4. The sensor of claim 1, wherein the first non-conductive material and the second non-conductive material comprise at least one of a knitted structure, a woven structure, a braided structure, a knotted structure, absorbent fibers, moisture-transporting fibers, and cotton fibers.

5. The sensor of claim 1, wherein the first outer sheath is in contact with the second outer sheath.

6. The sensor of claim 1, further comprising:
a third textile assembly having a third conductive element and a third outer sheath that surrounds the third conductive element, the third outer sheath formed from a third non-conductive material that is configured to transport moisture through the third outer sheath, the third conductive element being grounded,
the third outer sheath maintaining a separation between the third conductive element and the first and second conductive elements along the length of the sensor.

7. The sensor of claim 6, further comprising an outer cover that at least partially encases the first, second, and third textile assemblies.

8. A sweat level monitoring system comprising:
a sensor comprising:
a first textile assembly having a first conductive element and a first outer sheath that surrounds the first conductive element, the first outer sheath formed from a first non-conductive material, and
a second textile assembly having a second conductive element and a second outer sheath that surrounds the second conductive element, the second outer sheath formed from a second non-conductive material, the first and second sheaths maintaining separation between the first conductive element and the second conductive element,
the sensor configured to detect a voltage value between the first conductive element and the second conductive element; and
a processor configured to:
receive the detected voltage value from the sensor, and
map the detected voltage value to a moisture characteristic.

9. The sweat level monitoring system of claim 8, further comprising:
a memory configured to:
store a table that includes a plurality of voltage values and a plurality of moisture characteristics, wherein each of the plurality of voltage values is associated with a respective one of the plurality of moisture characteristics, and
wherein the moisture characteristic to which the detected voltage is mapped is one of the plurality of moisture characteristics.

10. The sweat level monitoring system of claim 9, wherein the table further includes a plurality of perceived moisture values, wherein each of the plurality of perceived moisture values is associated with a respective one of the plurality of moisture characteristics and a respective one of the plurality of voltage values.

11. The sweat level monitoring system of claim 10, further comprising:
a human-machine-interface (HMI) configured to receive a perceived moisture value input by a user, the input perceived moisture value being associated with the detected voltage,
wherein the processor is further configured to:
compare the input perceived moisture value to a respective one of the plurality of perceived moisture values to which the detected voltage is mapped, and
provide an indication to the user, via the HMI, if the input perceived moisture value is different from the respective one of the plurality of perceived moisture values.

12. The sweat level monitoring system of claim 11, wherein the plurality of perceived moisture values is input into the table via the HMI.

13. A method for determining a moisture characteristic with a moisture characteristic monitoring system, the method comprising:
detecting, via a sensor, a voltage value, wherein the sensor comprises a first textile assembly and a second textile assembly, the first textile assembly having a first conductive element and a first outer sheath that surrounds the first conductive element, the first outer sheath formed from a first non-conductive material, and the second textile assembly having a second conductive element and a second outer sheath that surrounds the second conductive element, the second outer sheath formed from a second non-conductive material, wherein the detected voltage value is detected between the first conductive element and the second conductive element; and
determining the moisture characteristic based on the detected voltage value.

14. The method of claim 13, wherein determining the moisture characteristic includes mapping the detected voltage value to the moisture characteristic stored in a table in a memory of the monitoring system, the table including a plurality of voltage values and a plurality of moisture values, wherein each of the plurality of voltage values is associated with a respective one of the plurality of moisture characteristics.

15. The method of claim 14, wherein the moisture characteristic is a first moisture characteristic and wherein the voltage value is a first voltage value, the method further comprising:
detecting, via the sensor, a second voltage value;
measuring a second moisture characteristic that corresponds to the detected second voltage value; and
storing, in the table in the memory, the second moisture characteristic associated with the second voltage value.

16. The method of claim 15, wherein the first voltage value is detected at a first time, and wherein the second voltage value is detected at a second time, the first time being different from the second time.

17. The method of claim 14, wherein the table further includes a plurality of perceived moisture values, wherein each of the plurality of perceived moisture values is associated with a respective one of the plurality of moisture characteristics and a respective one of the plurality of voltage values, the method further comprising:
receiving a perceived moisture value;
comparing the input perceived moisture value to a respective one of the plurality of perceived moisture values to which the detected voltage is mapped; and
indicating if the input perceived moisture value is different from the respective one of the plurality of perceived moisture values.

18. The method of claim 13, further comprising:
determining a resistance (Rₓ) between the first conductive element and the second conductive element based on the detected voltage value,
wherein the first conductive element is spaced apart from the second conductive element by a distance (d), wherein determining the moisture characteristic includes determining a surface area (S) of moisture on the first and second conductive elements based on a relationship defined by: Rₓ=R·d/S, wherein R is the resistivity of the first and second conductive elements.

19. A method, comprising:
with a first textile assembly having a first conductive element and a first outer sheath that surrounds the first conductive element, the first outer sheath formed from a first non-conductive material that is configured to transport moisture through the first outer sheath; and
a second textile assembly having a second conductive element and a second outer sheath that surrounds the second conductive element, the second outer sheath formed from a second non-conductive material that is configured to transport moisture through the second outer sheath,
the first and second outer sheaths maintaining a separation between the first conductive element and the second conductive element along a length of the sensor,
detecting a voltage related to an amount of moisture that places the first conductive element into electrical communication with the second conductive element.

20. The method of claim 19, further comprising mapping the voltage to a moisture characteristic stored in a table, the table including a plurality of voltage values and a plurality of moisture characteristics, wherein each of the plurality of voltage values is associated with a respective one of the plurality of moisture characteristics.

21. The method of claim 20, wherein the moisture characteristic comprises a volume, an accumulation, a rate of accumulation, a level of electrolyte, an evaporation, a rate of evaporation.
